(19) United States
(12) Patent Application Publication
(10) Pub. No.: US 2012/0321511 A1
(43) Pub. Date: Dec. 20, 2012

(54) GASEOUS CHLORINE DIOXIDE DECONTAMINATION SYSTEM AND METHOD

(76) Inventor: Paul W. Lorcheim, Lebanon, NJ (US)

(21) Appl. No.: 13/163,808

(22) Filed: Jun. 20, 2011

(51) Int. Cl. A61L 2/20; B23P 17/04; B01J 7/00

(52) U.S. Cl. 422/30; 422/162; 29/428

(57) ABSTRACT

A system includes a portable source of gaseous chlorine dioxide (CD) to be generated within an isolated chamber or tent structure enclosing the device to be treated. A portable scrubber has first sourced couplings for sealingly connecting to a scrubbing flow path comprising at least one gas conduit for removing the CD from the device enclosed within an isolated chamber or tent structure. The gas conduit may have second couplings for connecting the device or isolated chamber or tent structure to the scrubbing flow path by way of a connection panel to complete a closed, filtered gas loop.

### FIELD OF THE INVENTION

The present invention relates to decontamination systems and methods.

BACKGROUND

Class I or Class II (laminar flow) biological safety cabinets are designed to minimize hazards inherent in work with biological agents. BSC's can be used for work with biological agents assigned to biosafety levels 1 through 4, depending on the facility design as described in the CDC/NIH publication Biosafety in Microbiological and Biomedical Laboratories. A BSC is a ventilated device for personnel, product, and environmental protection having an open front with inward airflow for personnel protection, downward HEPA filtered laminar airflow for product protection, and REM filtered exhausted air for environmental protection.

Class III BSC's or isolators are similar to Class I or II but incorporate glove ports.

Recommendations and requirements to certify BSC's come from a variety of sources. All manufacturers and NSF International recommend field certification of BSC's. The Center for Disease Control (CDC) and NIH state that it is essential that Class I, II and Ill BSC's be tested and certified.

Decontamination is a key component of certification and/or maintenance activities.

### SUMMARY

In some embodiments, a system comprises a portable source of gaseous chlorine dioxide (CD) located in an enclosure. An isolated chamber or a flexible tent to form a sealed enclosure for containing a device to be treated with the CD.

In some embodiments, a system comprises a portable source of gaseous chlorine dioxide (CD) located in an enclosure. The source has first couplings for sealingly connecting a portable scrubber connecting to a scribing flow path comprising at least one gas conduit for removing the CD from the device.

In some embodiments, a system comprises a portable source of gaseous chlorine dioxide (CD) located in an enclosure. The source has first couplings for sealingly connecting a portable scrubber connecting to a scrubbing flow path comprising at least one gas conduit for removing the CD from the device. The gas conduit has second couplings for connecting the device to the scrubbing flow path to create a closed scrubbing loop.

In some embodiments, a system comprises a portable source of gaseous chlorine dioxide (CD) located in an enclosure. The source has first couplings for sealingly connecting the building exhaust system or HVAC exhaust system to at least one gas conduit for removing the CD from the device. The source has second couplings connecting to the connection panel, which allows for fresh air to enter and replace the CD gas surrounding the device.

In some embodiments, a method comprises forming a flexible tent film material or solid isolated enclosure to form a gas-tight enclosure around a device to be treated and joining this to at least one panel having fittings for connecting to gas conduits. The gas conduits are connected to the fittings and to couplings of a portable charcoal scrubber. A source of gaseous chlorine dioxide (CD) is located in the enclosure, so as to form a sealed CD generation chamber wherein the CD generating reaction occurs. Gaseous CD is generated from the source within the chamber to treat the device.

### BRIEF DESCRIPTION OF THE DRAWINGS

FIG. 1A is a schematic diagram of one embodiment, during operation in a ClO₂ generation mode.

FIG. 1B is a schematic diagram of system of FIG. 1A, during operation in a ClO, open scrubbing mode.

FIG. 1C is a schematic diagram of system of FIG. 1A, during operation in a ClO, closed scrubbing mode.

FIG. 1D is a schematic diagram of the system if FIG. 1A, during operation in a ClO, building exhaust/HVAC exhaust mode.

FIG. 2 is a view of the connection panel.

FIG. 3A is a view of the scrubber assembly of FIG. 1A.

FIG. 3B is a view of the scrubber assembly of FIG. 1B.

FIG. 4 is a view of the CD Generation Equipment of FIG. 1A.

FIG. 5 is a top level flow diagram of a method of using the system of 1A.

FIG. 6 is a flow chart of the chamber preparation process of FIG. 5.

FIG. 7 is a flow chart of the exhaust/aeration preparation process of FIG. 5.

FIG. 8 is a flow chart of intake preparation process solids of FIG. 5.

FIG. 9 is a flow chart of determining the amount of CD chemicals required of FIG. 5.

FIG. 10 is a flow chart of the CD generation preparation process of FIG. 5.

FIG. 11 is a flow chart of the decontamination cycle of FIG. 5.

FIG. 12 is a flow chart of the scrubbing cycle of FIG. 5.

FIG. 13 is a flow chart of the neutralization process of FIG. 5.

FIG. 14 is a flow chart of the post-decontamination procedure of FIG. 5.

### DETAILED DESCRIPTION

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

One embodiment provides an apparatus and method to produce a gas for gaseous decontamination to reduce microorganisms thereon by treating a device (such as a BSC) or item(s) in a temporary enclosed sealed space or chamber. The apparatus includes a gas generating apparatus and a means for removing the gas that is comprised of supply and return ducting with couplings connected to inlet and outlet ports affixed to respective connection panels incorporated onto the device or temporary enclosed sealed space. The apparatus has a closed chamber of gas-tight construction isolated from the ambient space.

The corresponding embodiment of a method comprises exposing the device or item(s) in a temporary enclosed sealed space or chamber to an atmosphere comprising gaseous chlorine dioxide. It may comprise controlling the concentration and required time cycles of chlorine dioxide generation, dwell and rapid removal. This embodiment of a method also provides the proper humidity to enhance the susceptibility of microorganisms and/or sporicidal action of chlorine dioxide. Neutralizer is added to the residual waste liquid within the apparatus, following which the waste liquid may be discarded by conventional or future developed means. The chlorine dioxide gas is produced from a precursor A and B solid chemical, mixed into an aqueous solution, then transformed to a gaseous state. The method may also be used with larger devices or sealed spaces with additional quantities of Chlorine Dioxide generating chemicals. Additional items may be added to the space under decontamination.

Embodiments may take physical form in certain parts and arrangement of parts, a preferred embodiment of is described in detail below and illustrated in the drawings.

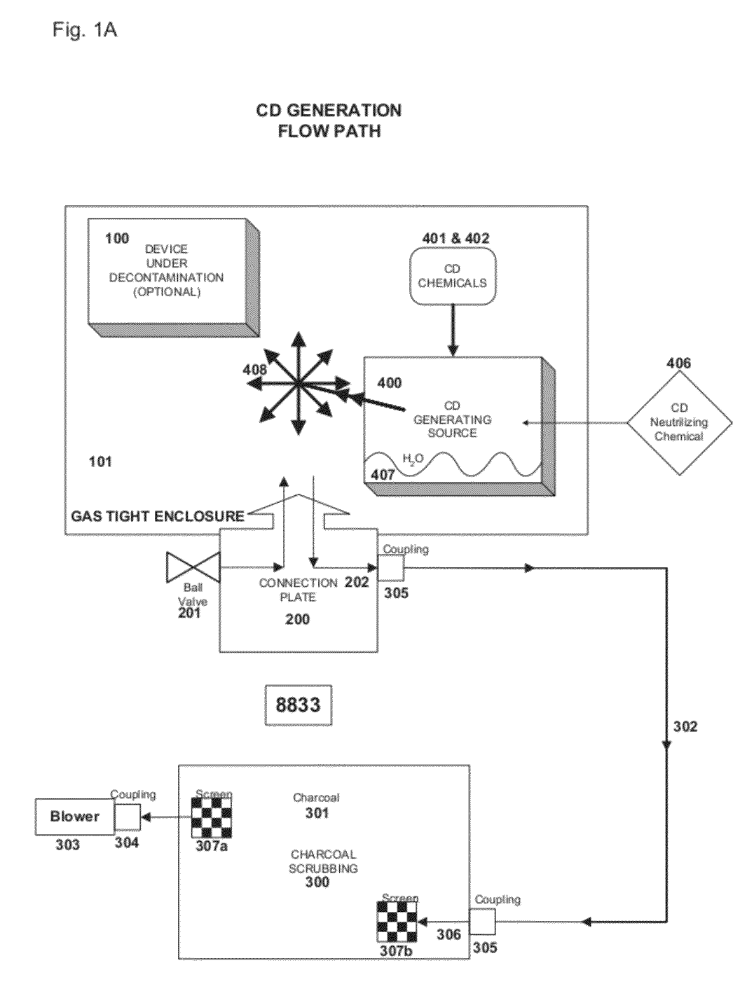

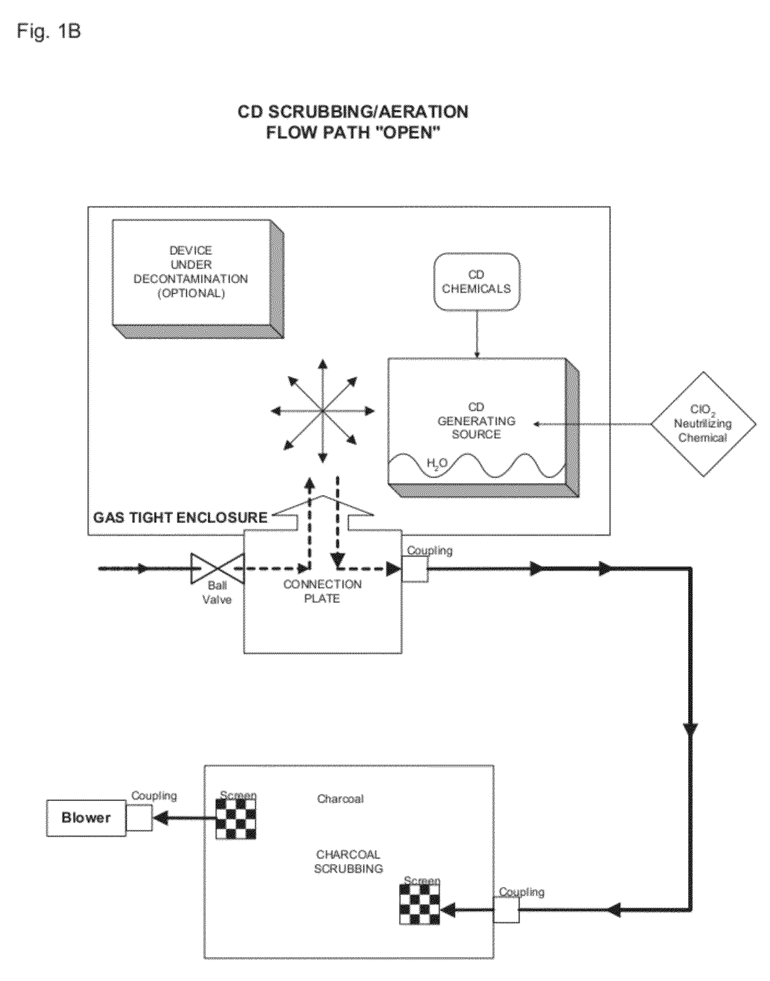

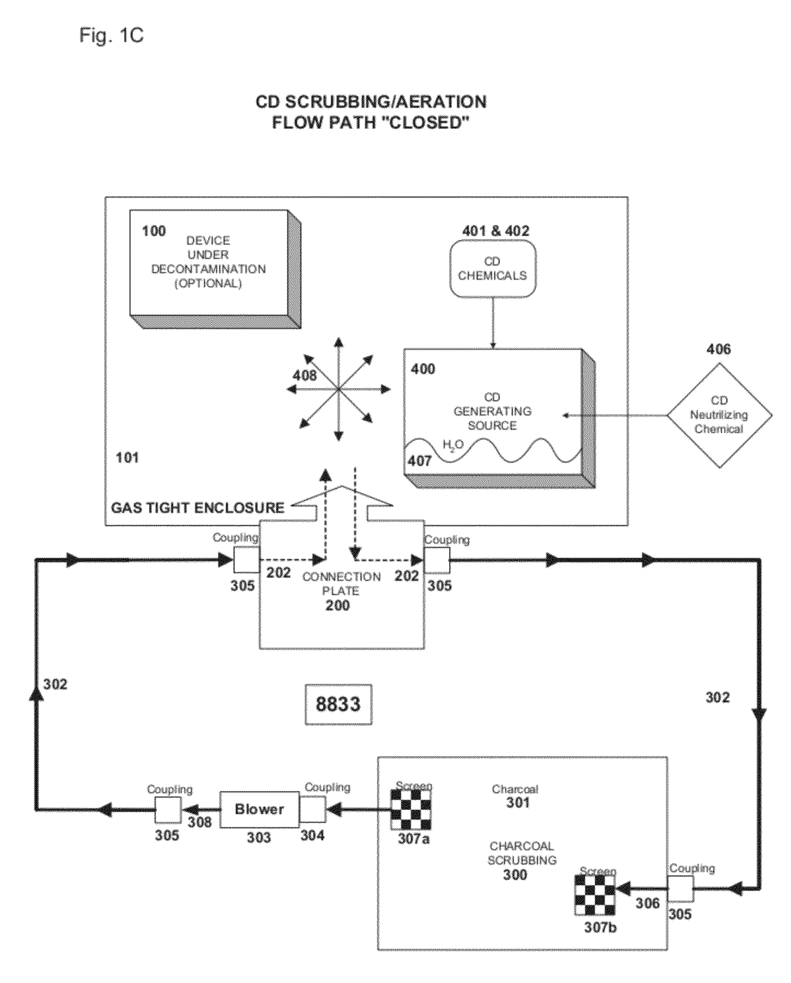

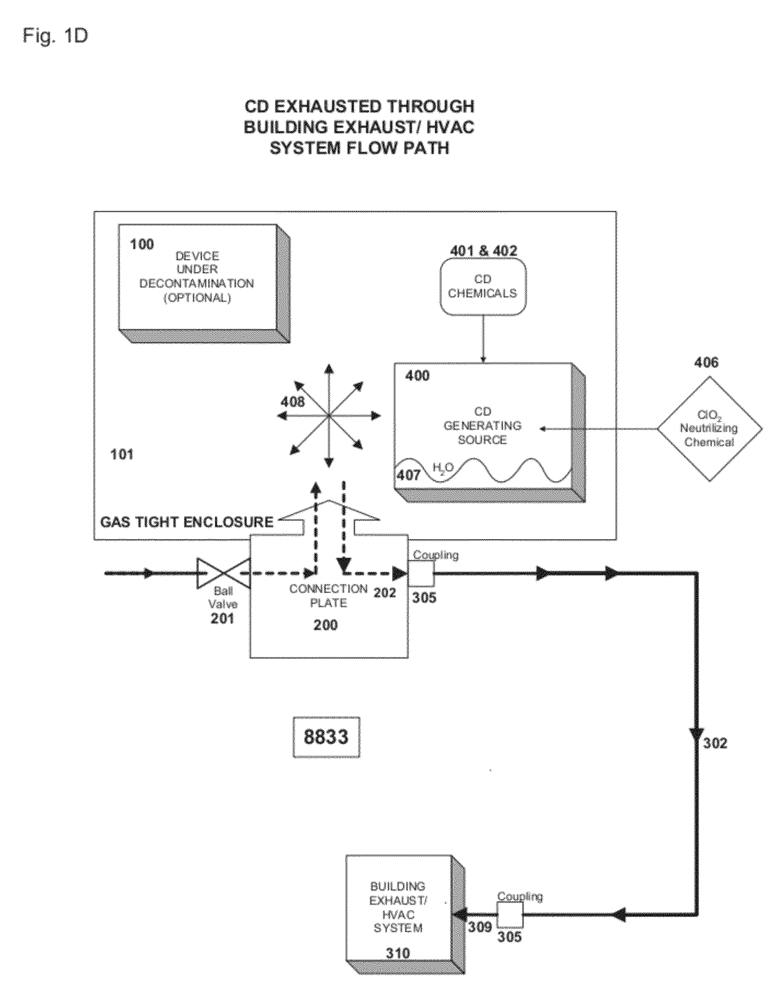

FIGS. 1A, 1B, 1C and 1D are schematic views of a Chem CD decontamination system 8833. The Chem CD 8833 system includes connection panel 200, a scrubber 300, and a source 400. The system is capable of being operated in a chlorine dioxide (CD or ClO₂) generation mode or in a scrubbing mode. The CD generation mode flow path is shown in FIG. 1A. The open scrubbing mode flow path is shown in FIG. 1B. The closed scrubbing mode flow path is shown in FIG. 1C. The building exhaust/HVAC exhaust mode flow path is shown in FIG. 1D.

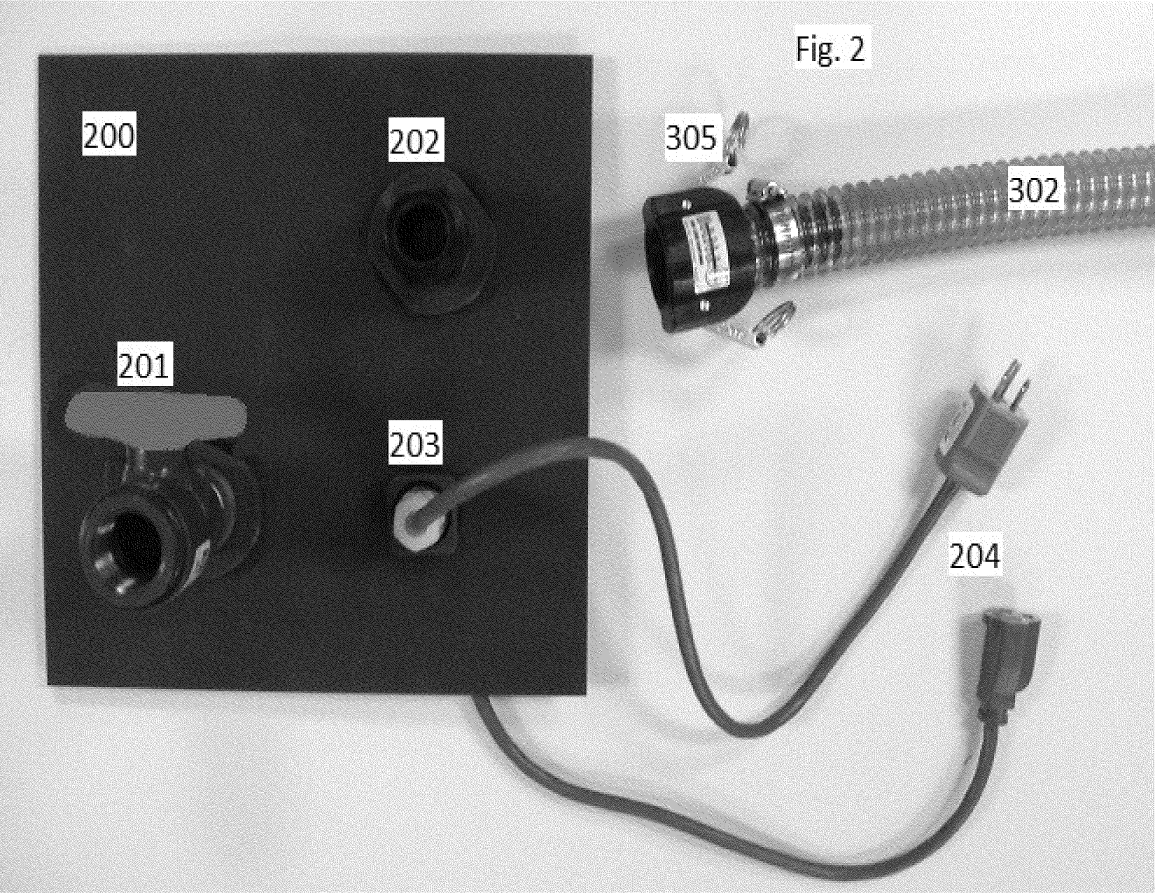

FIG. 1A shows a device under decontamination 100. In other embodiments, the device under decontamination 100 is surrounded by a gas tight tent/enclosure 101, which is in turn connected to the connection panel 200 (FIG. 2).

An optional biological indicator (not shown) can be placed within the decontamination zone. The device under decontamination 100 is then sealed, incorporating into the connection panel 200 (FIG. 2) a inlet 201 and outlet port 202 for use with the Chem CD 8833 system. In some embodiments, to accomplish the seal, the tent material is taped using a suitable pressure sensitive adhesive tape (such as duct tape) to a connection panel 200 (FIG. 2) having the inlet port 201 and/or outlet port 202. After an appropriate set up has been achieved, Chlorine Dioxide (ClO₂) is produced and released and the decontamination cycle begins. FIG. 1B, 1C and 1D show the apparatus with the CD scrubbing flow paths activated. After a suitable exposure time specified by NSF (e.g. 85 minutes), ClO₂ gas is removed from the device under decontamination 100 using one of the scrubbing cycles of the Chem CD 8833 system. After a suitable scrubbing time (e.g., approximately 45 minutes), the Chem CD 8833 system is neutralized and the device under decontamination 100 and the gas tight enclosure 101 may be unsealed.

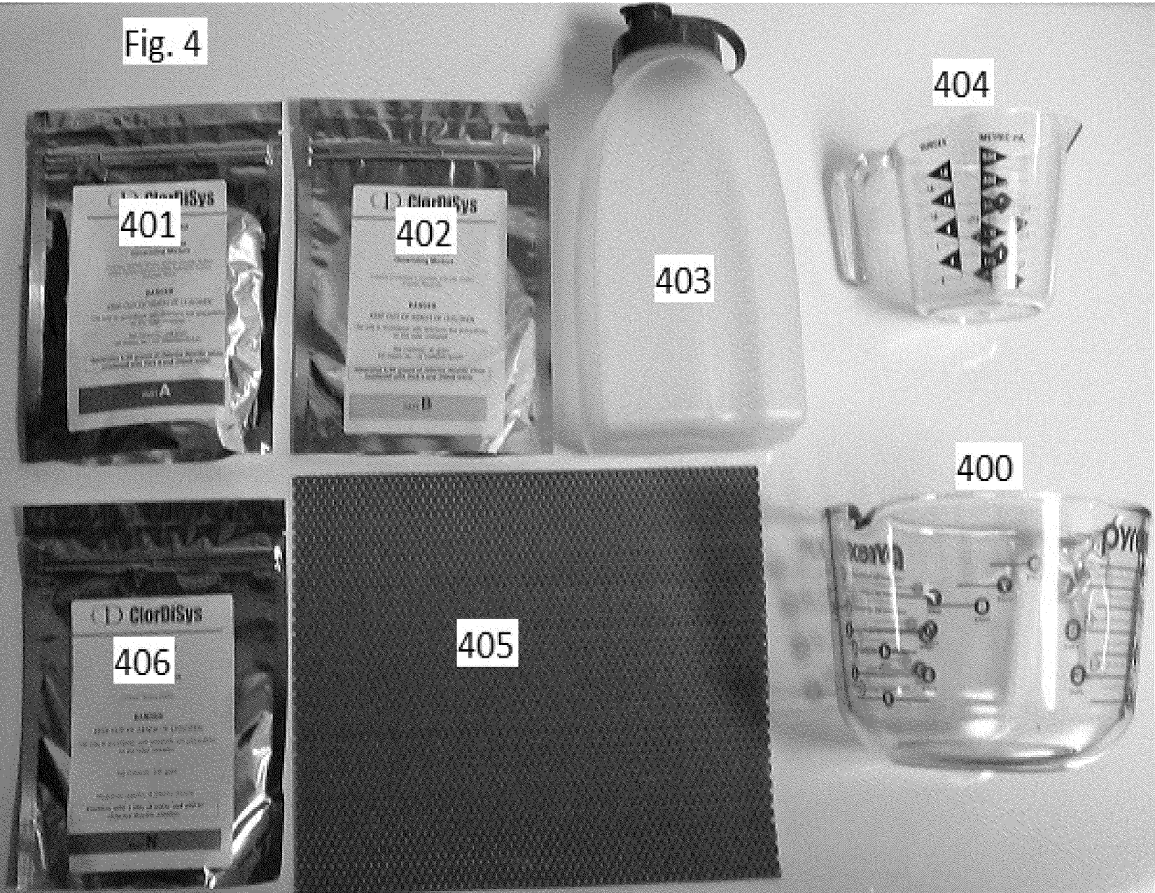

FIGS. 1A, 1B, 1C and 1D show a Chem CD decontamination system 8833 using chlorine dioxide (ClO₂) gas. The Chem CD 8833 system and the equipment (FIG. 4) is placed within a temporary enclosed sealed space 101 under decontamination. By way of example, and not limited to, a temporary enclosed sealed space 101 may take the form of a Class II type A1, A2, B1, and B2 biological safety cabinet (BSC), Class I BSC, Class III BSC, negative or positive isolators, animal devices, incubators, refrigerators and freezers, room or any other potentially contaminated item(s). The Chem CD 8833 system may be used with devices or temporary enclosed sealed spaces having a volume of typically less than 120 ft³ (3.4 m³). The Chem CD 8833 system may also be used with larger devices or sealed spaces with additional quantities of ClO₂ generating chemicals. Additional items may be added to the space under decontamination.

System 8833 includes a “gas tight” system that is comprised of conduit connected between inlet port 201 and outlet port 202 of device 100 or temporary enclosed sealed space 101. In some embodiments to seal or affix to the device 100 or temporary enclosed sealed space 101 under decontamination, a connection panel 200 is provided. Quick disconnect couplings 304, 305 (which may include locking levers or other positive sealing mechanisms) connect the lines from the scrubbing loop to the connection panel 200 and to the Chem CD 8833 system. CD Generation Flow Path (represented by the bolded arrows) FIG. 1A: ClO, generation source 400, is placed within the gas tight enclosure 101. CD chemical (CD precursor A) 401 is added to the CD generating source 400 and then 250 ml of water 407 is added. Then CD chemical (CD precursor B) 402 is added to CD chemical (CD precursor A) 401, the CD generating source 400, and 250 ml of water 407 resulting in the generation of ClO, gas.

ClO₂ open scrubbing path (represented by bolded arrows) FIG. 1B: The charcoal scrubber 300 is place outside the gas tight enclosure 101. The blower 303 is located within the open scrubbing loop including the supply ducting 302, attachment couplings 304, 305, 306, screens 307a, 307b, charcoal 301 and Chem CD 8833 system. The ClO₂ scrubbing blower 303 is driven by a motor (not shown). The blower 303 is sized to provide a high airflow volume to quickly scrub the CIO₂ gas from the device under decontamination 100.

ClO₂ closed scrubbing path (represented by bolded arrows) FIG. 1C: The charcoal scrubber 300 is place outside the gas tight enclosure 101. The blower 303 is located within the closed scrubbing loop including the supply ducting 302, the scrubber 300, attachment couplings 304, 305, 306, screens 307a, 307b, charcoal 301 and Chem CD 8833 system. The ClO₂ scrubbing blower 303 is driven by a motor (not shown). The blower 303 is sized to provide a high airflow volume to quickly scrub the ClO₂ gas from the device under decontamination 100.

ClO₂ building exhaust/HVAC exhaust path (represented by bolded arrows) FIG. 1D: The building exhaust./HVAC exhaust system 310 is attached to the connection panel 200 including the supply ducting 302, attachment couplings 305, 309 screens and building exhaust/HVAC exhaust system 310. The ClO₂ scrubbing blower 303 is driven by a motor (not shown). The building exhaust/HVAC exhaust system 310 is sized to provide a high airflow volume to quickly scrub the ClO₂ gas from the device under decontamination 100.

A ClO₂ generator within the Chem CD 8833 system includes a CD generating source/reservoir 400, CD precursor A 401, CD precursor B 402, measuring cup 404, and a porous splash guard 405. ClO₂ is generated within CD generating source/reservoir 400 and is contained within the temporary enclosed sealed space 101.

FIG. 2 shows a connection panel 200 that is incorporated in the scrubbing loop and allows the charcoal scrubber to attach to the gas tight temporary enclosure 101. Within the connection panel 200 there are inlet 201 and outlet ports 202 allowing the scrubber to remove the ClO₂ from the gas tight temporary enclosure 101. In some embodiments the connection panel may have a electricity source port 203 with electricity cord 204 to have the option to power an electrical device within the gas tight temporary enclosure during the decontamination cycle.

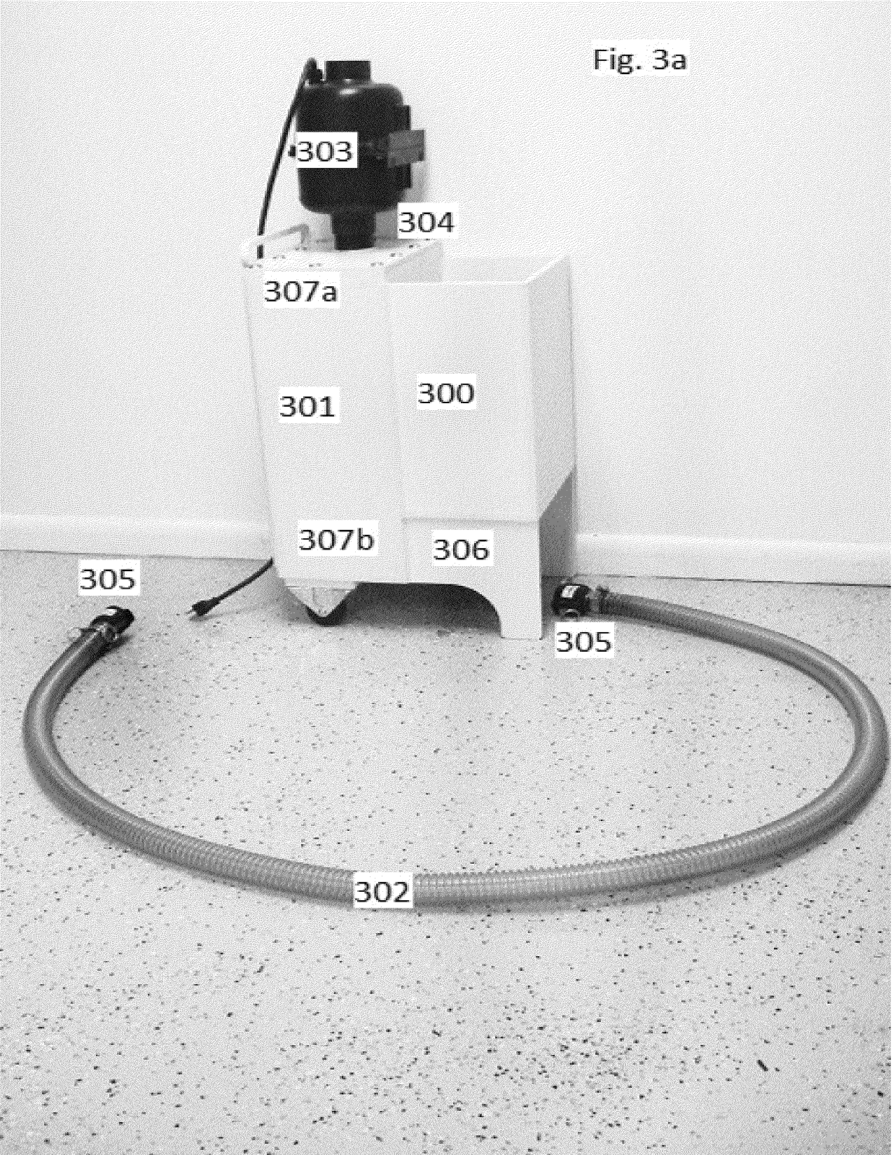

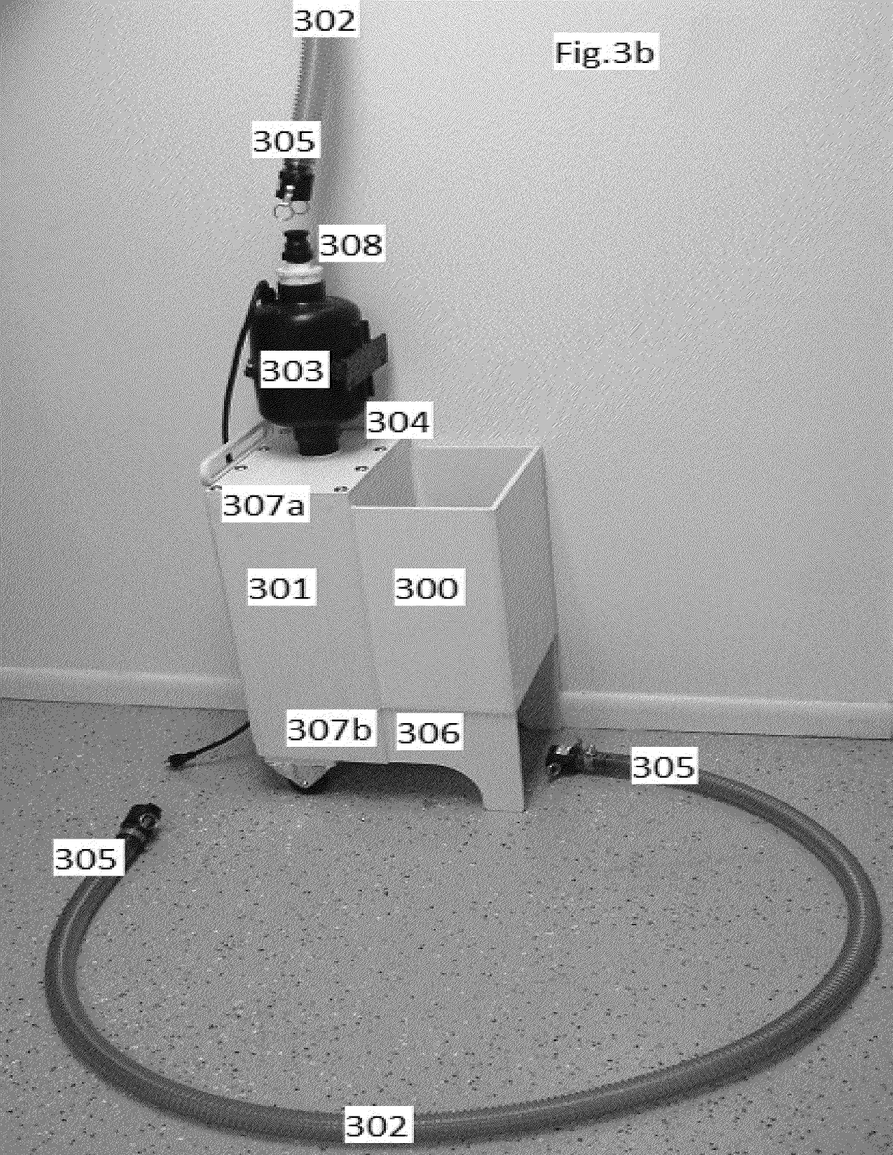

FIG. 3A shows a ClO₂ charcoal scrubber 300 including, conduit 302 and fitting 305 and 306 for attaching to the connection panel 200 and the scrubber. ClO₂ charcoal scrubber 300 includes an inlet screen 307b, outlet screen 307a with associated conduit 302, and fittings 305 attaching conduit 302 wherein ClO₂ is removed from the device 100 or temporary enclosed sealed space 101. The ClO₂ charcoal scrubber 300 includes a blower 303 and coupling 304 to attach to the charcoal scrubber 300, with associated conduit 302 to draw the ClO2 gas into the charcoal bed 301 and exhausting out the top creating a open scrubbing path.

FIG. 3B shows a ClO, charcoal scrubber 300 including, conduit 302 and fitting 305 and 306 for attaching to the connection panel 200 and the scrubber. ClO₂ charcoal scrubber 300 includes an inlet screen 307b, outlet screen 307a with associated conduit 302, and fittings 305 attaching conduit 302 wherein ClO, is removed from the device 100 or temporary enclosed sealed space 101. The ClO₂ charcoal scrubber 300 includes a blower 303 and coupling 304 to attach to the charcoal scrubber 300, with associated conduit 302 to draw the ClO₂ gas into the charcoal bed 301. In some embodiments the blower may include a coupling 308 allowing conduit 302 to attach with coupling 302 to the blower to create a closed scrubbing path or to attach to a building exhaust/HVAC exhaust system.

FIG. 4 shows the CD Generation and neutralization equipment and chemicals. FIG. 4 includes the CD generating reservoir 400, CD precursor A 401, CD precursor B 402, neutralizing precursor N mixing bottle 403, measuring cup 404, porous splash guard 405 and neutralizing precursor N 406,

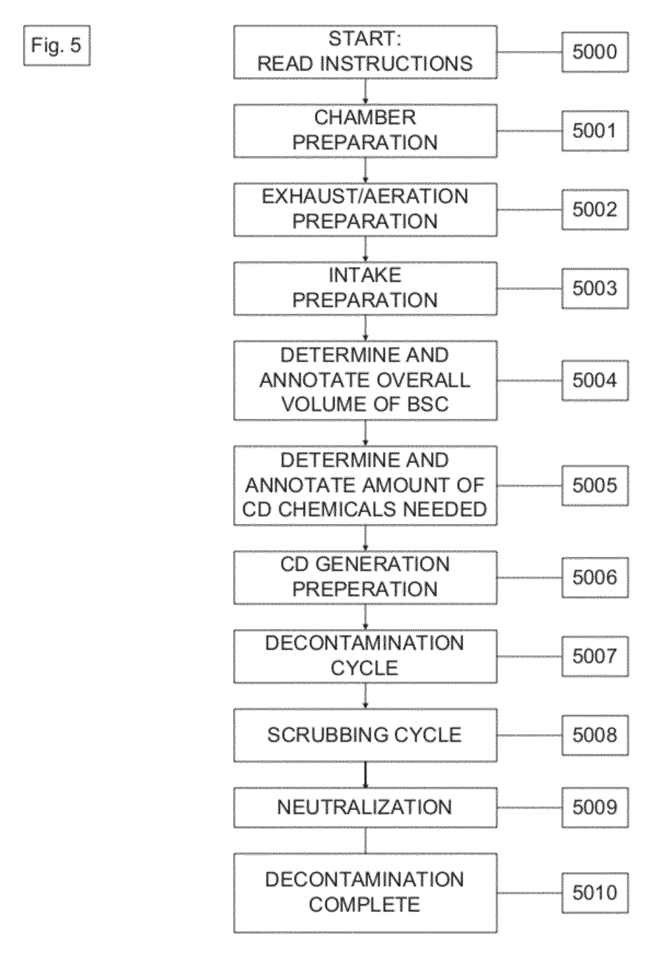

FIG. 5 is a high level flow chart of a process for performing a decontamination of a Class II Type A1, A2, B1, and B2 Biological Safety Cabinet (BSC) 101. When the system of FIGS. 1A, 1B, 1C and 1D is applied to other devices, slight modifications on attachment, sealing and circulation are applied, as will be apparent to one of ordinary skill. For example, in other embodiments, the device to be decontaminated is not a BSC, and a tent material is placed around the device, and sealed to appropriate connection panels described herein, using a pressure sensitive adhesive tape (e.g., duct tape). The gas conduit connections to the ports of the connection panels can be made in the same manner as connecting the conduit to the BSC. In addition, a power cord 204 for the running an electrical device can be passed through an opening or fitting 203 in the connection panel 200 and a gas-tight seal formed around the cord.

Referring again to FIG. 5, at step 5000, prior to using the system, the user reviews the manual and safety procedures.

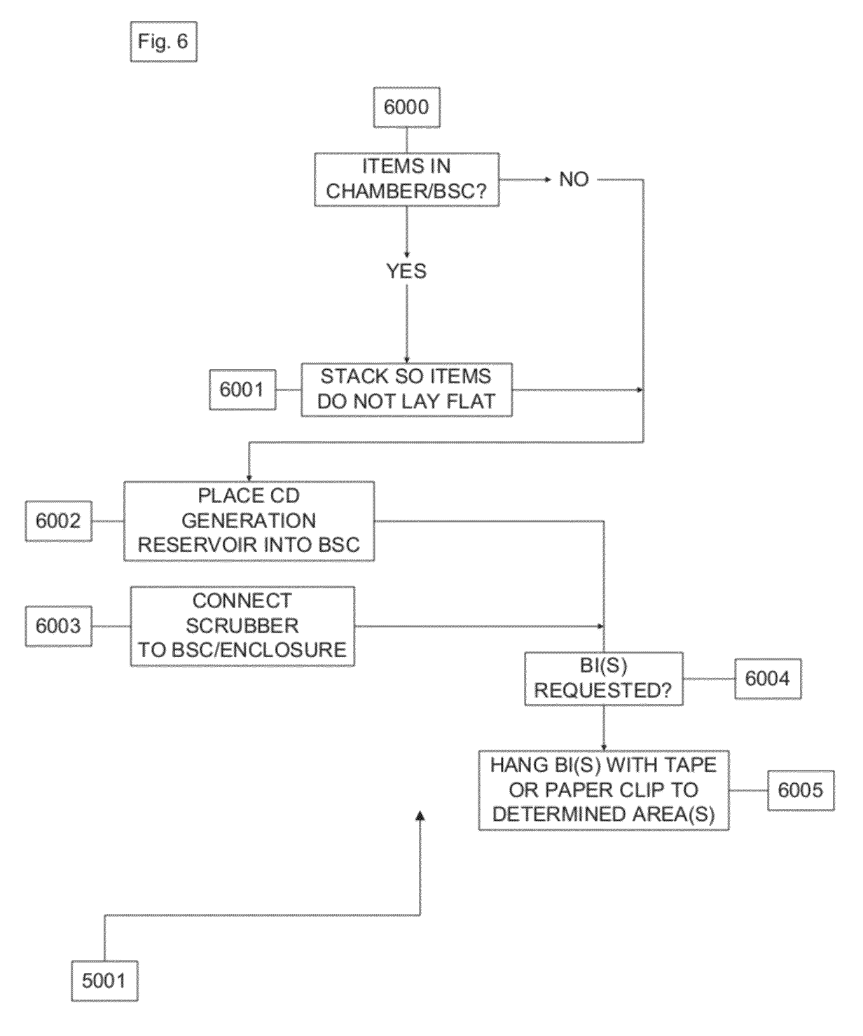

At step 5001, the BSC 101 is prepared. Details of this step are discussed below with reference to FIG. 6.

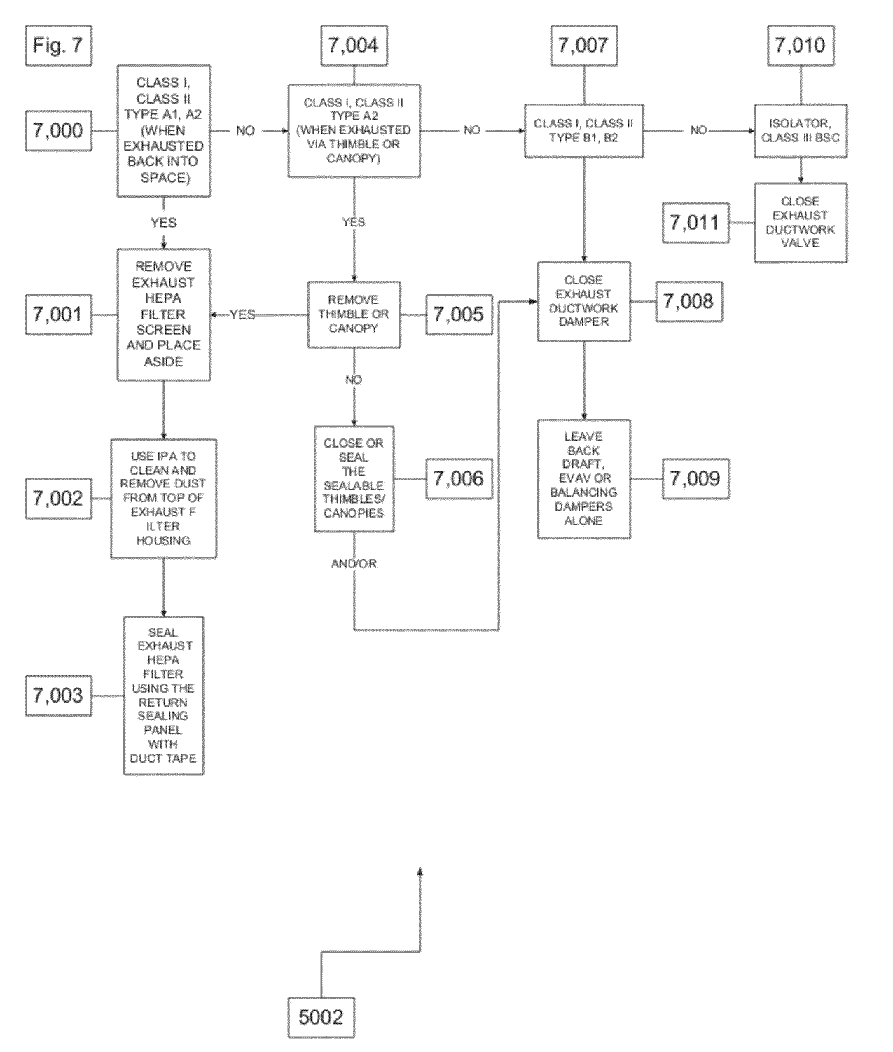

At step 5002, the exhaust/aeration preparations are performed. Details of this step are discussed below with reference to FIG. 7.

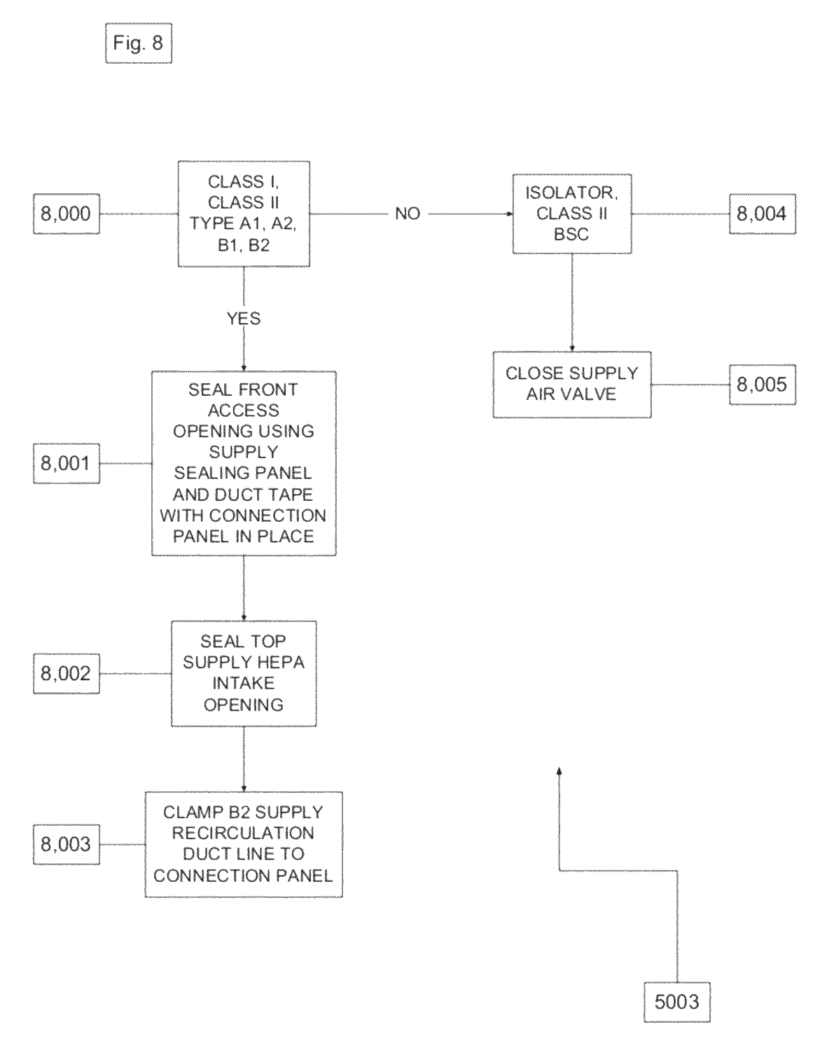

At step 5003, the intake preparations are performed. Details of this step are discussed below with reference to FIG. 8.

At step 5004, the overall volume of BSC 101 is determined and annotated.

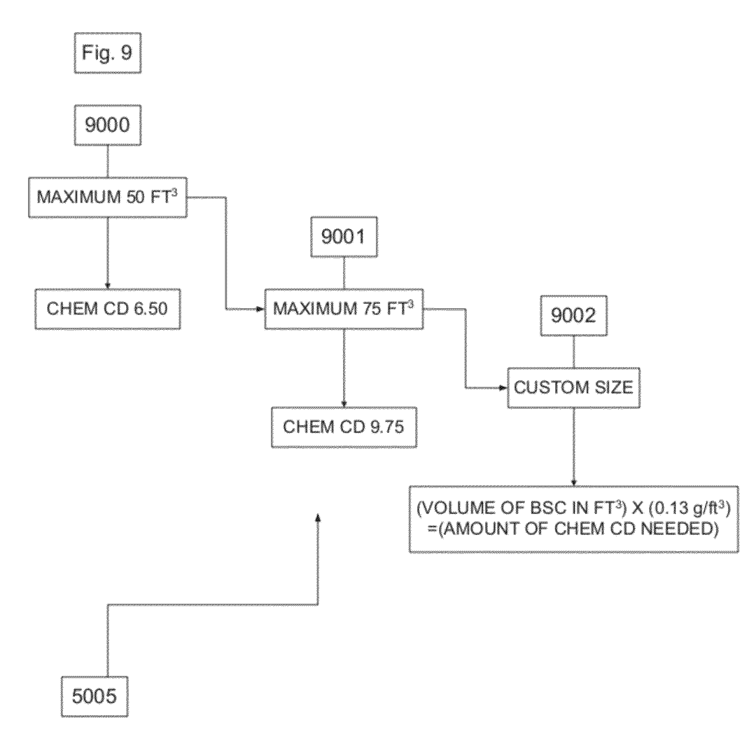

At step 5005, the amount of CD precursor is determined and noted. Details of this step are discussed below with reference to FIG. 9.

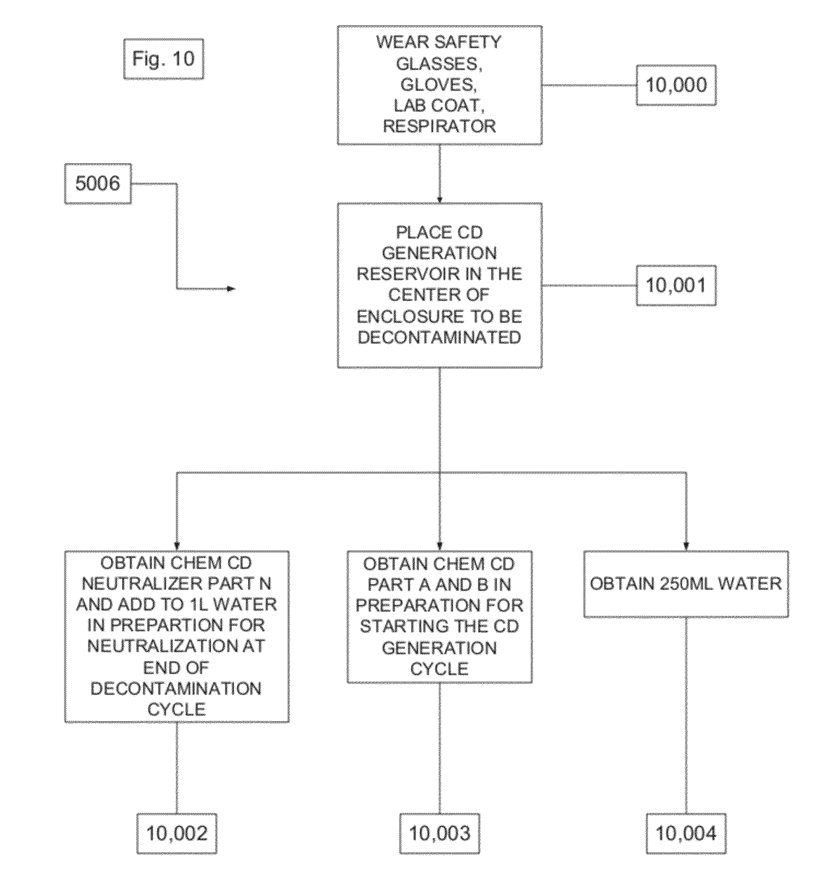

At step 5006, the Chem CD 8833 system is prepared. Details of this step are discussed below with reference to FIG. 10.

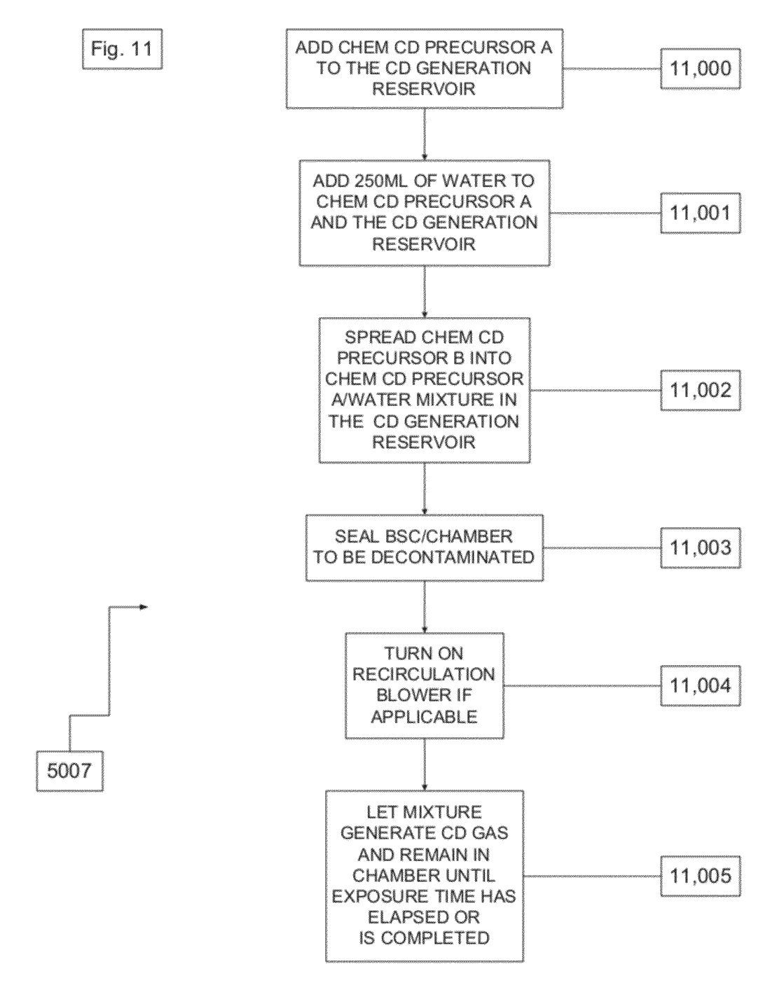

At step 5007, the decontamination cycle is performed. Details of this step are discussed below with reference to FIG. 11.

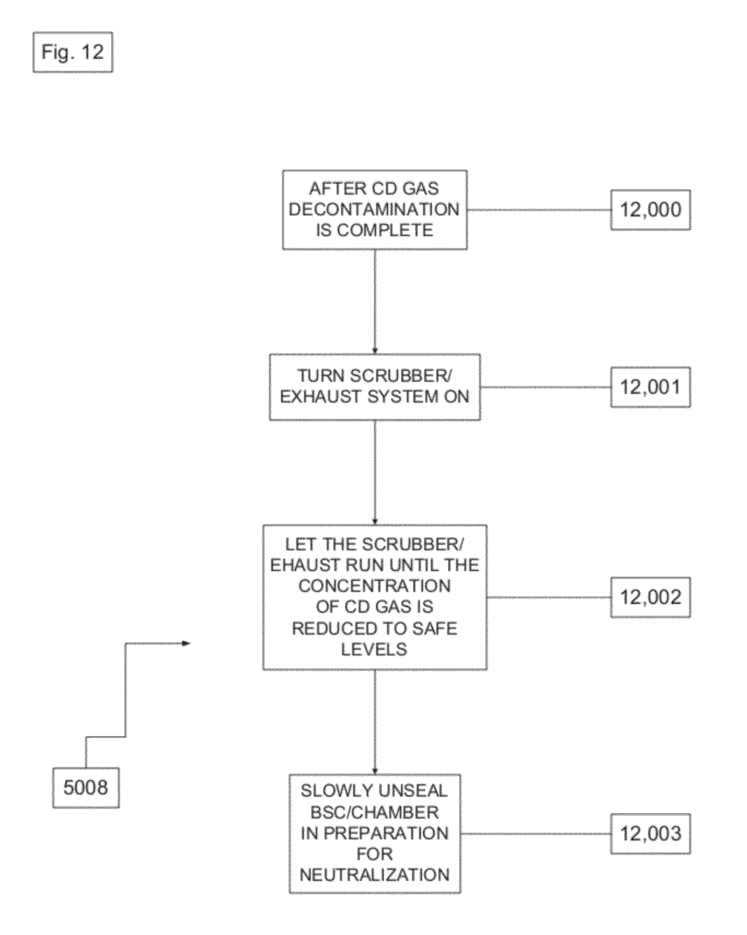

At step 5008, the scrubbing cycle is performed. Details of this step are discussed below with reference to FIG. 12.

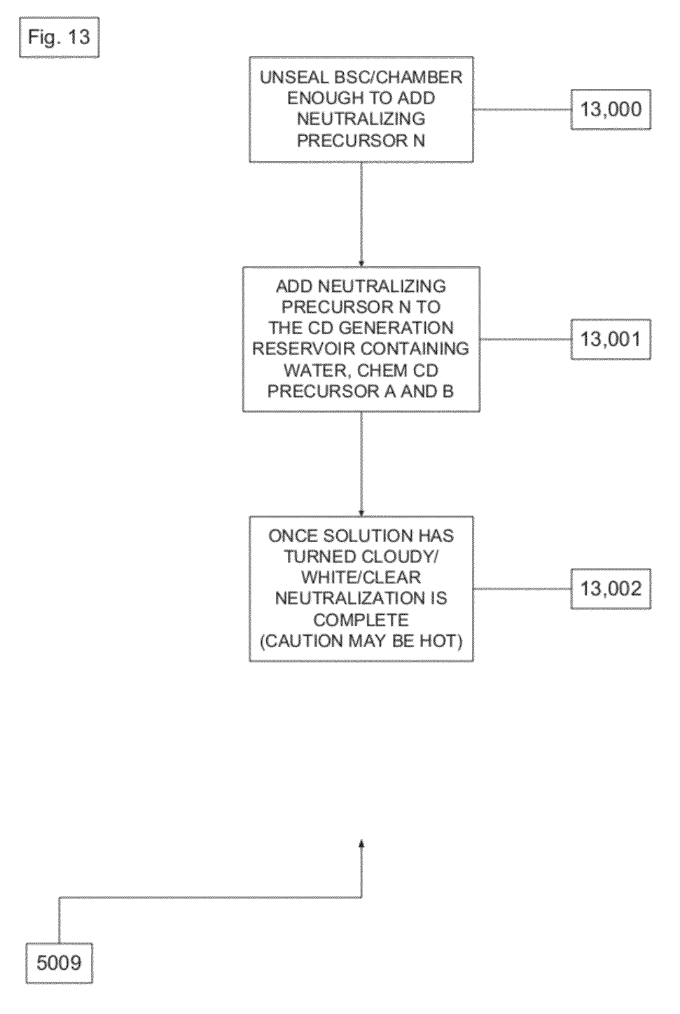

At step 5009, the neutralization step is performed. Details of this step are discussed below with reference to FIG. 13.

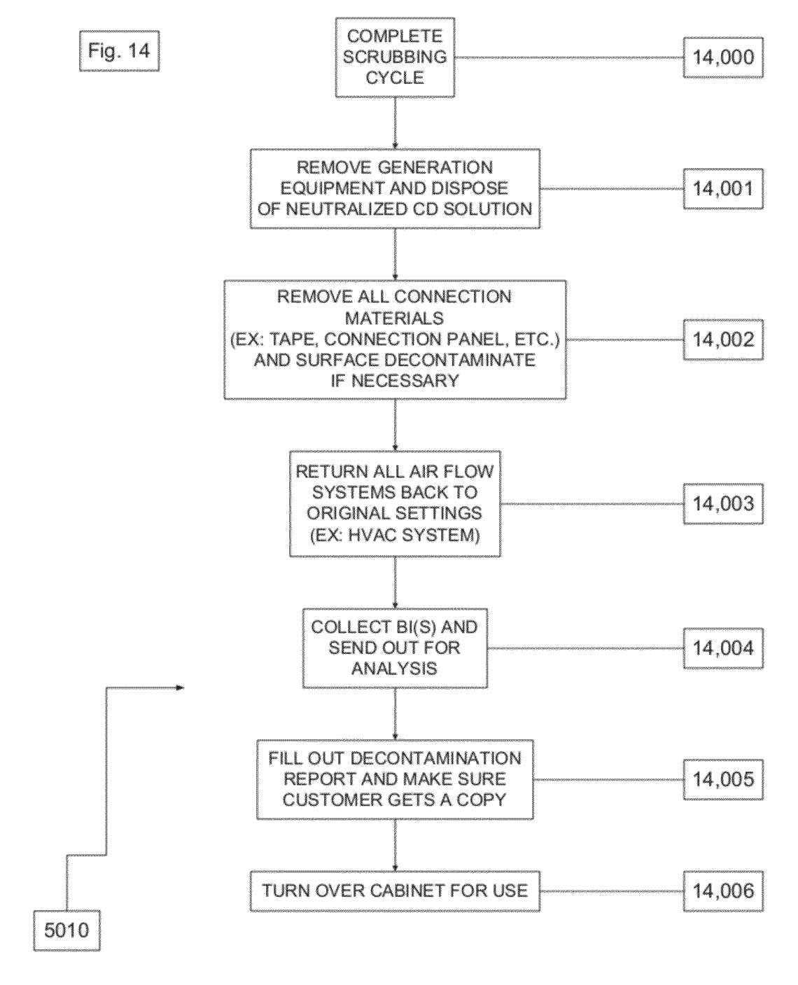

At step 5010, the decontamination is complete, and a post-decontamination procedure is performed. Details of this step are discussed below with reference to FIG. 14.

Referring to FIG. 6, the BSC Preparation is shown.

At step 6000, the user verifies that only items to be decontaminated are within the BSC.

At step 6001, the user verifies that all items remaining in the BSC are stacked in a way that the humidity and ClO₂ gas can contact all surfaces, and no items lay flat or obstructed. If items require power, they are plugged into the BSC's receptacle and tested for operation ensuring the current draw does not exceed the rated capacity of the BSC's receptacle(s). The BSC may be prepped or moved such that appropriate sealing will be possible (e.g., in animal areas where the BSC units are on casters).

At step 6002, the user places the CD generation reservoir into the BSC or temporary gas tight enclosure.

At step 6003, the user connects the charcoal scrubber to the BSC or temporary gas tight enclosure.

At step 6004, a determination is made whether a biological indicator (BI) was requested.

At step 6005, if requested, the user can optionally affix at least one biological indicator (BI) within the BSC(s) at a pre-determined location(s). If using a BI with a Tyvek envelope, the user pushes a hanger (e.g., an opened paperclip or other hanger) through one end of the Tyvek envelope, and then attaches the hanger to an internal surface of the BSC. If the hanger cannot be directly hung, the surface is decontaminated with the appropriate disinfectant or sterilant, ensuring proper contact time prior to affixing the tape.

FIG. 7 shows the exhaust preparation step. Exhaust preparation of various classifications use different exhaust sealing configurations, as follows:

At step 7000, steps 7001-7003 are performed for Class I, Class II Type A1, A2 when the air through the BSC is exhausted back into the space.

At step 7001, the user removes the exhaust HEPA filter protective screen and places it aside.

At step 7002, the user uses isopropyl alcohol (IPA) or other cleaning solvents to clean and remove dust or debris from the top exhaust filter housing.

At step 7003, the user seals the exhaust HEPA filter using a return sealing panel (which may be configured with male cam and groove coupling) using duct tape or other sealing material.

At step 7004, steps 7005-7006 are performed for Class I, Class II Type A2 (when exhausted via thimble or canopy).

At step 7005, the user removes the thimble or canopy. Thimble or canopy connections are spelled out in the National Sanitation Foundation (NSF International) Standard No. 49 for Class II (Laminar Flow) Biohazard Cabinetry, for connecting BSC to exhaust systems. This type of connection provides an air gap as to compensate for room pressurization changes.

Alternatively, at step 7006, the user closes or seals the sealable type thimbles, canopies, and/or at step 626, the user closes the exhaust gas tight damper and follows the B1 or B2 procedure dependant on the sealing, and or, damper location relative to the exhaust HEPA filter. The user ensures that this is indeed a gas tight damper with no by-pass leakage. The user temporarily by-passes any low flow alarms.

At step 7007, steps 7008-7009 are performed for a Class I, Class II Type B1, B2 BSC.

At step 7008, the user fully closes the exhaust ductwork gas tight decontamination exhaust damper.

At step 7009, the user leaves the back draft, EVAV or other balancing damper(s) in their original position.

At step 7010 steps 7011 are used for isolators or class III BSC's.

At step 7011, the user closes the exhaust ductwork valve.

FIG. 8 is a flow chart showing intake preparation of classifications. Steps 8000-8003 are performed for a Class I, Class II Type A1, A2, or B1 BSC. Steps 8004-8005 are performed for Isolators and Class II Type B2 BSC.

At step 8000, preparation starts for a Class I, Class II Type A1, A2, or B1 BSC.

At step 8001, the user starts to seal the front access opening using the supply connection panel using duct tape or other sealing materials.

At step 8002, the user seals the top supply HEPA intake opening.

At step 8003, the user clamps the B2 supply recirculation duct line to the connection panel.

At step 8004, preparation starts for a isolator or a Class II BSC.

At step 8005, the user closes the air supply valve.

FIG. 9 shows the process by which the user determines and annotates the amount of ClO, generating chemicals need to be used for the decontamination.

The user multiplies the BSC volume by 0.13 g/ft³ (4.7, g/ m³) to determine the mass of ClO₂ required to be generated. Then, the user multiplies the ClO₂ mass by the unit mass of the supplied chemical. The following table determines the amount of chemical (e.g., sodium chlorite) required. Need 0.13 g CD/ft³ of space being decontaminated. For example: a 6 foot BSC is 75 ft³.times.0.13=9.75 CD required.

TABLE-US-00001
TABLE 1 Chlorine Dioxide Minimum Maximum BSC Size Generating
Table 1

| Maximum | BSC Size | Chlorine Dioxide |
|---|---|---|
| Volume - ft³ (m³) | Width - ft (m) | Generating Chemical (g) |
| 50 | 3-4 ft (0.91-1.22) | 6.5 |
| 75 | 5-6 ft (1.52-1.83) | 9.75 |
| Custom | Custom | (Volume ft³) × (0.13 g/ft³) |

FIG. 10 shows the Chem CD System Preparation.

At step 10,000, the user dons safety glasses, lab coat, gloves and an appropriate respirator.

At step 10,001, the user places the CD generation reservoir in the center of the enclosure or BSC to be decontaminated.

At step 10,002, the user obtains Chem CD neutralizer precursor N 406 and adds IL of water to mixing bottle 403 in preparation for neutralization at the end of the decontamination cycle.

At step 10,003, the user obtains Chem CD precursor A 401 and Chem CD precursor B 402 in preparation for starting the CD generation cycle.

At step 10,004, the user obtains 250 ml of water 407 using the measuring cup 404.

FIG. 11 shows the decontamination Cycle. Before beginning the cycle, the user verifies that a negative pressure secondary containment system is incorporated within the decontamination area‘3or that the BSC is located within an un-recirculated space with a pressure negative relative to all bordering areas, labs and hallways, etc.

At step 11,000, the user adds the Chem CD precursor A 401 to the CD generation source/reservoir 400.

At step 11,001, the user adds 250 ml of water 407 using the measuring cup 404.

At step 11,002, the user spreads Chem CD precursor B 402 into Chem CD precursor A 401 and 250 ml of water 407 in the CD generation source/reservoir 400.

At step 11,003, the user will quickly seal the BSC or temporary gas tight enclosure 101 to be decontaminated.

At step 11,004, the user activates the recirculation blower if applicable.

At step 11,005, the user lets the mixture generate ClO₂ gas and remain in the chamber until the exposure time has elapsed or is complete.

FIG. 12 is a flow chart of the Scrubbing Cycle

At step 12,000, the user determines that the ClO₂ gas contact cycle is complete.

At step 12,001, the user turns on the scrubber 300 or building exhaust/HVAC exhaust system 310.

At step 12,002, the user lets the scrubber 300 or building exhaust/HVAC exhaust 310 run until the concentration of ClO₂ gas is reduced to safe levels.

At step 12,003, the user slowly unseals the BSC 101 or temporary gas tight enclosure 101 in preparation for neutralization.

FIG. 13 shows the neutralization procedure.

At step 13,000, the user makes sure to unseal the BSC 101 or temporary gas tight enclosure 101 just enough to add the neutralizing precursor N 406 after it has been mixed in IL of water using the mixing bottle 403.

At step 13,001, the user adds the neutralizing precursor N 406 after it has been mixed in 1 L of water using the mixing bottle 403 to the CD generation source/reservoir containing the 250 ml of water 407, CD precursor A 401 and CD precursor B 402.

At step 13,002, once the solution has turned cloudy/white/clear then neutralization is complete (CAUTION: solution may be hot)..

FIG. 14 shows the final procedure performed when Decontamination is Complete.

At step 14,000, the user monitors determines that the scrubbing cycle is complete..

At step 14,001, the user disassembles removes the generation equipment and disposes of the neutralized solution.

At step 14,002, the user removes all connection materials (ex: tape, connection panel, etc.) and surface decontaminates if necessary.

At step 14003, the user returns all air flow systems back to original settings (ex: HVAC system).

At step 14,004, the user collects the BI(s) if used and sends them out for analysis.

At step 14,005, the final decontamination report will be filled out completely and the user will make sure the customer gets a copy.

At step 14006, the user will turn the BSC 101 or temporary gas tight enclosure 101 over for use.

Many variations and options are may be included in various embodiments.

In some embodiments, a gas tight connection panel connector is provided for the return of the scrubbed gas. Gas tight sealing duct ports of various diameters may be used for the return of the scrubbed gas

In some embodiments, gas tight sealing duct caps of various diameters are provided for the return of the scrubbed gas.

In some embodiments, the neutralization powder may be a proprietary mixture.

In some embodiments, the charcoal scrubber box has an inlet incorporating a charcoal retention screen with a gas tight design.

Preferably, the piping design incorporates the one blower, charcoal scrubber box, and provisions to attach the inlet and outlet lines all incorporated into one system. The blower is for the scrubbing or removal of the ClO₂ gas.

The Chem CD 8833 allows one to provide complete decontamination services in less than 4 hours, including setup and tear down of Biological Safety Cabinets (BSC) or devices (e.g., Casework, Cabinets, HLF's or VLF's, Containment Devices, CFH's, Centrifuges, Refrigerators, Freezers, Washers, Water Baths, Shakers, Bio-reactors, Tanks, Ctrs, Computers, or any other lab or productions equipment).

Other Items may be incorporated within the decontamination space, and can be placed within the BSC.

Examples of BSC's which the Chem CD 8833 is compatible with, are all classes and type classifications.

Some embodiments include a connection panel with a port to introduce a power cord to energize an electrical device within the device or space under decontamination.

A gas tight connection panel may be included for “tenting method” or temporary spaces to contain the gas for the introduction of the decontaminating gas. Similarly, a gas tight sealing duct port of various diameters may be included for scrubbing the gas from a BSC (Type B2).

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

1. A system comprising: a portable source of gaseous chlorine dioxide (CD), the source to be placed within an isolated chamber or flexible tent; so as to form a sealed enclosure for containing a device to be treated with the CD; and the CD generation source.

2. The system of claim 1, wherein the source includes: an open reservoir able to withstand the CD generating reaction; a first container separated from the reservoir for adding a solid or powder CD precursor A to the reservoir; a second container separated from the reservoir for adding water to the reservoir and the CD precursor A; a third container separated from the reservoir for adding a solid or powder CD precursor B to the reservoir, CD precursor A and the water.

3. The system of claim 2, wherein the first container includes a solid or powder dispensing container.

4. The system of claim 3, wherein the first container includes: a package for containing CD precursor A until addition of the CD precursor A into the open reservoir.

5. The system of claim 2, wherein the second container includes a water measuring and dispensing container.

6. The system of claim 5, wherein the second container includes: water measuring and dispensing container for containing water until addition of water to CD precursor A into the open reservoir.

7. The system of claim 2, wherein the third container includes a solid or powder dispensing container.

8. The system of claim 7, wherein the third container includes: a package for containing CD precursor B until addition of CD precursor A and water to the reservoir to start the CD generating reaction.

9. The system of claim 1, further comprising of connections to a building exhaust system (HVAC) for removing CD from the device.

10. The system of claim 9, comprising of couplings for sealingly connecting at least one gas conduit from the enclosure to the building exhaust system (HVAC) exhausting the CD gas, thus removing it from the device.

11. The system of claim 9, comprising of couplings for sealingly connecting at least one gas conduit from environment to the device allowing for fresh air to replace CD contaminated air surrounding the device during exhaustion.

12. The system of claim 11, further comprising of couplings for sealingly connecting a ball valve to allow fresh air to replace CD gas surrounding the device.

13. The system of claim 1, further comprising a portable scrubber for removing CD from the isolated chamber or flexible tent, the scrubber having couplings for sealingly connecting to a scrubbing flow path comprising at least one gas conduit for removing the CD from the device.

14. The system of claim 13, further comprising a scrubber having couplings for sealingly connecting to a scrubbing flow path comprising at least one gas conduit for removing the CD from the device and another gas conduit for returning filtered air to the device sealingly through a connection panel or from the surrounding environment.

15. The system of claim 13, further comprising: a blower in the scrubbing flow path for pumping or pulling the CD through the scrubber.

16. The system of claim 13, wherein the scrubber includes a charcoal filter through which the CD in the scrubbing flow path is to be pumped to or pulled from, thus being absorbed and removed from the air.

17. The system of claim 13, wherein the scrubber includes charcoal particles through which the CD is filtered and removed from the airstream.

18. The system of claim 17, wherein the scrubber includes: a charcoal container, and a screen coupled to the scrubbing flow path for dispersing the CD through the charcoal container.

19. The system of claim 17, wherein the scrubber includes: a charcoal container, and a screen coupled to the scrubbing flow path for containing the charcoal particles within the scrubber.

20. The system of claim 13, wherein the scrubbing flow path includes gas-tight couplings connecting the scrubber to the source; or the connection panel.

21. The system of claim 20, wherein the connection panel is connected to the CD scrubber flow path.

22. The system of claim 21, wherein the scrubbing flow path includes a check valve for preventing backflow when the scrubber is not in use.

23. The System of claim 1, further comprising: a solid porous splash guard cover able to withstand the CD generating reaction.

24. The System of claim 23, further comprising: a solid splash guard cover acting as a prevention system so as to prevent the CD generating reaction from making contact with the device.

25. The system of claim 1, further comprising having a neutralizing chemical CD precursor N that can be added the CD precursor A, B and water after the device is treated; and the CD is removed from the air; using a mixing container for dispensing.

26. The system of claim 25, wherein the adding includes having a mixing container for mixing 1 L of water with a neutralizing chemical CD precursor N.

27. The system of claim 25, wherein the adding includes having mixing containers for adding the neutralizing chemical CD precursor N solution to the reservoir, CD precursor A, B and water after the decontamination and scrubbing is complete.

28. A method comprising: using a portable source of gaseous chlorine dioxide (CD) located in the enclosure, a portable scrubber having first couplings for sealingly connecting to a scrubbing flow path comprising at least one gas conduit for removing the CD from the device, the gas conduit having second couplings for connecting the device to the scrubbing flow path.

29. The method of claim 28, wherein the source includes: an open reservoir, a first container for adding a solid or powder CD precursor A to the reservoir; a second for adding water to the reservoir and the CD precursor A; a third container for adding a solid or powder CD precursor B to the reservoir, CD precursor A and the water; a porous splash guard cover, to prevent splashing while still releasing gaseous CD; a portable charcoal scrubber filled with charcoal particles; and a fourth container for adding a neutralizing chemical CD precursor N to the CD precursor A, B and water after the decontamination and scrubbing is complete.

30. The method of claim 28, further comprising: using a solid porous splash guard cover able to withstand the CD generating reaction.

31. The method of claim 30, further comprising: using a solid splash guard cover to act as a prevention system so as to prevent the CD generating reaction from making contact with the device.

32. The method of claim 28, further comprising: adding a solid or powder CD precursor A, B and water to an open reservoir within the source; beginning the CD generating reaction emitting the gaseous CD.

33. The method of claim 32, wherein the adding includes using containers for dispensing a solid or powder CD precursors A and B.

34. The method of claim 28, further comprising adding a neutralizing chemical CD precursor N to the CD precursor A, B and water after the device is treated; and the CD is removed from the air; using a mixing container for dispensing:

35. The method of claim 34, wherein the adding includes using mixing containers for mixing 1 L of water with a neutralizing chemical CD precursor N.

36. The system of claim 35, wherein the adding includes using mixing containers for adding the neutralizing chemical CD precursor N solution to the reservoir, CD precursor A, B and water after the decontamination and scrubbing is complete.

37. A method comprising: joining an isolator chamber or a flexible tent material to a connection panel having fittings for connecting to source and/or return conduits; so as to form a gas-tight enclosure around a device to be treated; so as to form a sealed chamber wherein the CD generation reaction occurs, with the portable source of gaseous CD located within.

38. The method of claim 37, wherein the joining includes sealingly attaching the isolated chamber or tent to at least one connection panel using a pipe fitting or pressure sensitive adhesive tape.

39. The method of claim 37, further comprising connecting the isolated chamber or tent to a portable scrubber by way of a scrubbing flow path; and using the scrubber to remove and filter the CD from the isolated chamber or tent after the device is treated.

40. The method of claim 39, wherein said using the scrubber includes pumping or pulling the gas in the scrubbing flow path through a bed of charcoal particles.

41. The method of claim 37, wherein: a first blower in the CD scrubbing flow path is used for pumping or pulling gaseous CD from the isolated chamber or tent through the scrubber, filtering the CD from the air, then releasing the filtered air to the environment or returning filtered air to the enclosure surrounding the device.
